(19) United States
(12) Patent Application Publication
(10) Pub. No.: US 2004/0059522 A1
(43) Pub. Date: Mar. 25, 2004

(54) Method for partitioned layout of protein interaction networks

(76) Inventors: Kyungsook Han, Yeonsu-gu (KR); Yanga Byun, Nam-gu (KR)

Correspondence Address: Gregory P. LaPointe, BACHMAN & LaPOINTE, P.C., Suite 1201, 900 Chapel Street, New Haven, CT, 06510-2802, US

(21) Appl. No.: 10/290,433

(22) Filed: Nov. 7, 2002

(30) Foreign Application Priority Data
Sep. 23, 2002 (KR) 2002-0057603

(51) Int. Cl. G06F 19/00; G01N 33/48; G01N 33/50

(52) U.S. Cl. 702/19

(57) ABSTRACT

Disclosed is a method for partitioned layout of protein interaction networks into a three-dimensional graph, comprising the steps of grouping nodes into group 1, group 2 and group 3 based on their interaction properties; computing shortest paths between nodes of each group, between nodes of the group 1 and nodes of the group 2, between nodes of the group 1 and nodes of the group 3, and between nodes of the group 2 and nodes of the group 3; and layout drawing by positioning nodes of the group 3 in the center of a sphere, nodes of the group 2 in the outer region of the group 3, and nodes of the group 1 in the outer region of the groups 2 and 3, by spring-force layout algorithm. The present invention is advantageous in terms of a clear and aesthetically pleasing drawing and being much faster than other forced-directed layouts.

### BACKGROUND OF THE INVENTION

1. Field of the Invention

The present invention relates to a new method of visualizing protein interaction data into a three-dimensional graph, and more particularly, to a method of visualizing large-scale protein interaction data into a clear and aesthetically pleasing graph by classifying protein nodes into three groups.

2. Description of the Prior Art

Protein-protein interaction data is rapidly increasing in volume at an unpredictable rate. The interaction data is available in forms of text files or databases. Because of being large-scale, the data can be more easily understood when being expressed into graphs than a long list of interacting proteins. In this regard, active research to visualize protein interaction networks is underway.

However, when being visualized into an undirected graph, protein interaction data has features as follows: first, the data yields a complex non-planar graph with a large number of edge crossings that cannot be removed in a two-dimensional drawing; second, since proteins have a very wide range of interacting proteins within the same set of data, the undirected graph contains nodes of high degree as well as those of low degree; third, when visualized as a graph, the data yields a disconnected graph comprising many connected components, and the MIPS genetic interaction data (http://mips.gsf.de/proj/yeast/tables/interaction/) contains, for example, 113 connected components; fourth, the data often contains protein interactions corresponding to self-loops, in which a source node and a target node are identical.

Owing to the features of protein interaction data, the conventional graph-drawing tools are problematic in terms of having difficulty in performing interactive works with a large volume of data due to their very slow execution, drawing a confused graph with too many edge crossings, and yielding a static graph in which it is difficult to revise in order to reflect updated data.

Based on a relaxation algorithm, a Java Applet program was developed for visualization of protein interactions, which was tested on Y2H (yeast two-hybrid) data. However, this program has several disadvantages as follows. The program requires all protein interaction data to be provided as parameters of the Applet program in HTML sources. There is no way to save a visualized graph except by capturing the window. Also, images captured from the window are static and typically of low quality, and cannot be refined or changed later to reflect an update in data. Further, a user can move a node, but cannot select or save a connected component containing a specific protein for further use.

On the other hand, when carrying out some visualization works for protein interactions, not their own algorithms or programs developed for visualization, but general-purpose drawing tools are used. For example, PSIMAP displays interactions between protein families by comparing Y2H data with DIP data. PSIMAP was drawn by Tom Sawyer software (http://www.tomsawyer.com/) and then refined through extensive manual work to remove edge crossings. In view of graph drawing, PSIMAP is a static image and leaves many needs for improvement. A research group at University of Washington tried to visualize Y2H data using AGD (http://www.mpisb.mpg.de/AGD/), which is another general-purpose drawing tool. Because of being a general-purpose drawing tool, despite being powerful, AGD does not provide a function required for studying protein-protein interactions.

### SUMMARY OF THE INVENTION

To solve the problems encountered in the prior art, taking the features of protein interaction data, as described above, into consideration, it is an object of the present invention to provide a new force-directed layout algorithm visualizing protein interactions in a three-dimensional space. In more detail, the present invention aims to provide a method of visualize large-scale protein interaction data into a clear and aesthetically pleasing graph by dividing protein nodes into three groups based on their interaction properties, which is much faster than the conventional algorithms.

### BRIEF DESCRIPTION OF THE DRAWINGS

The above and other objectives, features and other advantages of the present invention will be more clearly understood from the following detailed description taken in conjunction with the accompanying drawings, in which:

FIG. 1 illustrates an example of a partitioned graph;

FIG. 2 describes algorithm FindCutvertex determining nodes of V₂;

FIG. 3 describes algorithm IsCutvertex determining whether a node is a cutvertex or not, which is called in the algorithm of FIG. 2;

FIG. 4 describes an algorithm finding shortest paths between every pair of nodes in each group;

FIG. 5 describes an algorithm finding shortest paths between every pair of nodes in each sub-group, which is called in the algorithm of FIG. 4;

FIGS. 6a to 6d illustrate a drawing process of MIPS physical interaction data; and

FIG. 7 is a graph comparing running times of the graph-drawing algorithm according to the present invention with those of two conventional algorithms.

### DESCRIPTION OF THE PREFERRED EMBODIMENTS

To achieve the above objectives, the present invention provides a method for grouping nodes into the following three groups:

group 1 (V₁) is a set of terminal nodes of degree 1,

group 2 (V₂) consists of nodes of V-V₁, which are in the subgraphs separated by cutvertices of degree >=3, except nodes in the largest subgraph, and

group 3 (V₃) consists of nodes which are members of neither group 1 nor 2.

The present invention also provides a method for computing shortest paths between nodes of each group, shortest paths between nodes of the group 1 and nodes of the group 2, shortest paths between nodes of the group 1 and nodes of the group 3, and shortest paths between nodes of the group 2 and nodes of the group 3; and performing layout by positioning nodes of the group 3 in the center of a sphere, nodes of the group 2 in the outer region of the group 3, and nodes of the group 1 in the outer region of the groups 2 and 3, by spring-force layout algorithm using said shortest paths.

Many algorithms for force-directed graph drawing are too slow when visualizing large-scale protein interactions. Therefore, the present invention intends to improve running time by presenting a new algorithm, which divides nodes into three groups based on their interaction properties. The layout provided by the present invention is an extension of Kamada & Kawai's algorithm. Kamada & Kawai's algorithm produces two-dimensional drawings only, but we modified their algorithm not only for three-dimensional drawings but also for improvements in the efficiency and resultant drawings thereof.

At first, refer to the grouping of nodes. Groups 1, 2 and 3 are represented by V₁, V₂ and V₃, respectively, below.

Protein interaction data can be visualized as an undirected graph G=(V,E), where nodes V represent proteins and edges E represent protein-protein interactions. The degree of node vᵢ is the number of its edges denoted by deg (vᵢ). An edge e=(vᵢ,vⱼ) with vᵢ=vⱼ is a self-loop. A cutvertex in a graph G is a node whose removal disconnects G. A path in a graph G is a sequence (v₁, v₂, . . . , vₙ) of distinct nodes of G, in which (vᵢ,vᵢ₊₁)εE for 1≦i≦n−1.

In accordance with the present invention, nodes are divided into three exclusive and exhaustive groups, V₁, V₂ and V₃. The three groups are defined as follows: (i) group V₁ is a set of terminal nodes, that is, nodes of degree 1; (ii) group V₂ consists of nodes of V-V₁, which are in the subgraphs separated by cutvertices of degree >=3, except nodes in the largest subgraph; and (iii) group V₃ consists of nodes which are members of neither group V₁ nor V₂.

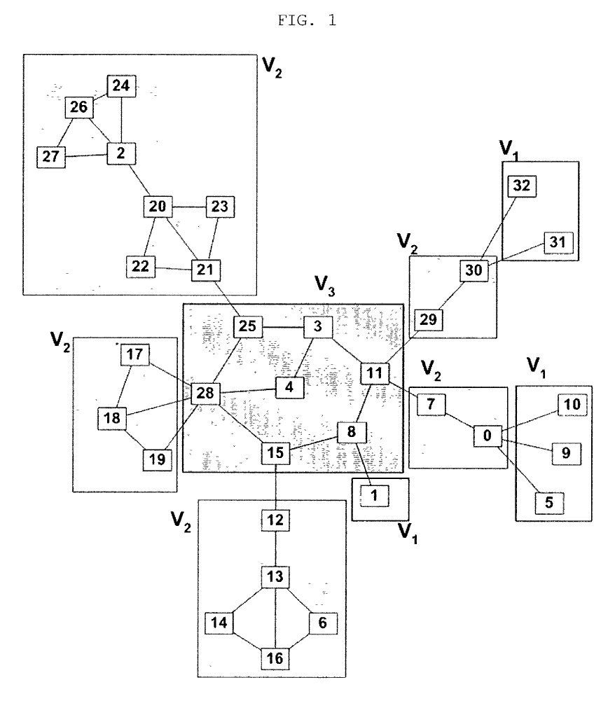

FIG. 1 shows an example of a partitioned graph, in which nodes in a graph G=(V,E) are separated into three groups. Six nodes belong to group V₁, and are separated into three sub-groups, V₁={{v₁}, {v₅, v₉, v₁₀}, {v₃₁, v₃₂}}. Each sub-group shares a neighboring node.

As shown in FIG. 1, because of sharing a cutvertex v₁₁, two sub-groups S₁={v₀, v₇} and S₂={v₂₉, v₃₀} are integrated into one sub-group of V₂. Sub-groups S₃={v₂₄, v₂₆, v₂₇} and S₄={v₂, v₂₀, v₂₁, v₂₂, v₂₃, v₂₄, v₂₆, v₂₇} do not share a cutvertex because the cutvertex of S₃ is v₂ and the cutvertex of S₄ is v₂₅. However, since the cutvertex of S₃ belongs to S₄, S₃ is merged into S₄ since S₃ is a subset of S₄.

Nodes of each group are found in the order of V₁, V₂ and V₃. First, nodes with one neighbor are classified into V₁, and nodes of V₁ are further divided into sub-groups according to their shared neighbors. Nodes of V₂ are then found from V-V₁, and all remaining nodes constitute V₃.

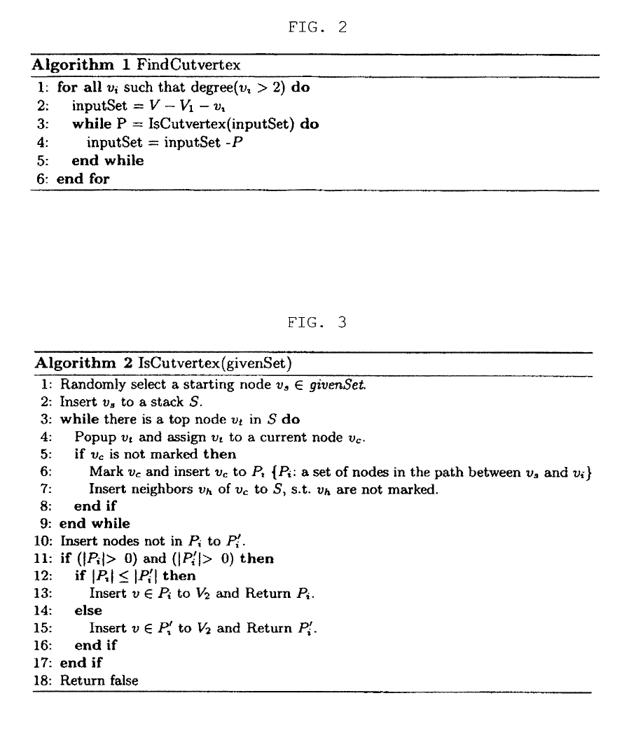

After finding V₁, nodes of V₂ are determined by FindCutvertex outlined in algorithm of FIG. 2. The initial input to the algorithm is nodes of V-V₁, and the algorithm tests whether the node is a cutvertex (line 3 in FIG. 2). Let P be the set of nodes in a path between vᵢ and the starting node, and P′ be the set of nodes not in the path. If neither P nor P′ is empty, the node vᵢ is a cutvertex, and the loop is repeated for the remaining nodes. The nodes in the smaller set between P and P′ are included in V₂ (lines 11-17 in FIG. 3). The nodes of V₂ are further separated into sub-groups based on their cutvertex, and the sub-groups are merged into one if they have the same cutvertex. After determining V₁ and V₂, all remaining nodes constitute V₃. Thus, V₃ corresponds to a biconnected subgraph (a connected graph with no cutvertex) in protein interaction data (herein, in case of a specific graph in which all nodes are connected in a line, V₃ is not a biconnected subgraph).

A forced-directed layout for three-dimensional graph drawing according to the present invention is as follows.

The algorithm by Kamada & Kawai, on which the present invention is based, searches for a drawing in which the energy is locally minimized. The algorithm according to the present invention focuses on finding a drawing in which an actual distance between two nodes is approximately proportional to a desirable distance between them. The global energy E of a spring system with n nodes is defined according to the following Equation 1:

$$\begin{matrix}E = {\sum }_{i = 1}^{n - 1} {\sum }_{j = i + 1}^{n} \frac{1}{2} {{k}_{ij} ( \vert {p}_{i} - {p}_{j} \vert - {l}_{ij} )}^{2} \\ = {\sum }_{i = 1}^{n - 1} {\sum }_{j = i + 1}^{n} \frac{1}{2} {k}_{ij} ⌈ {( {x}_{i} - {x}_{j} )}^{2} + {( {y}_{i} - {y}_{j} )}^{2} + {( {z}_{i} - {z}_{j} )}^{2} + \\ {l}_{ij}^{2} - 2 {l}_{ij} \sqrt{{( {x}_{i} - {x}_{j} )}^{2} + {( {y}_{i} - {y}_{j} )}^{2} + {( {z}_{i} - {z}_{j} )}^{2}} ⌉\end{matrix} \qquad [ Equation 1 ]$$

wherein, kᵢⱼ is a stiffness parameter of a spring, pᵢ is the position of a node vᵢ, and lᵢⱼ is the length of a spring connecting vᵢ and vⱼ.

The algorithm according to the present invention finds a position pₘ=(xₘ, yₘ, zₘ) for each vertex vₘ to minimize the potential energy in the spring system. As shown in Equation 2, below, the potential energy is minimized when the partial derivatives of E with respect to each variable xₘ, yₘ and zₘ are zero, giving a set of 3|V|=3n equations:

$$\frac{δ E}{δ {x}_{m}} = \frac{δ E}{δ {y}_{m}} = \frac{δ E}{δ {z}_{m}} = 0 , {v}_{m} ∈ V \qquad [ Equation 2 ]$$

In Kamada & Kawai's algorithm, a node is moved to a position to minimize energy while all other nodes remain fixed. The node to be moved is chosen as the one with the largest force acting on it, that is, the one for which Equation 3, below, is maximized over all vₘεV.

$$\sqrt{\frac{{( δ E )}^{2}}{δ {x}_{m}} + \frac{{( δ E )}^{2}}{δ {y}_{m}} + \frac{{( δ E )}^{2}}{δ {z}_{m}}} \qquad [ Equation 3 ]$$

However, this approach often produces undesirable graphs or requires too much time for large-scale protein interactions. Thus, the algorithm according to the present invention moves all nodes to some levels in each iteration until the difference between the current position and the previous position falls below a certain threshold value. For an initial layout, nodes are arranged on the surface of a sphere, instead of being placed randomly. Therefore, the algorithm according to the present invention yields more attractive drawings and is much faster for production of graphs with balanced groups than Kamada & Kawai's algorithm.

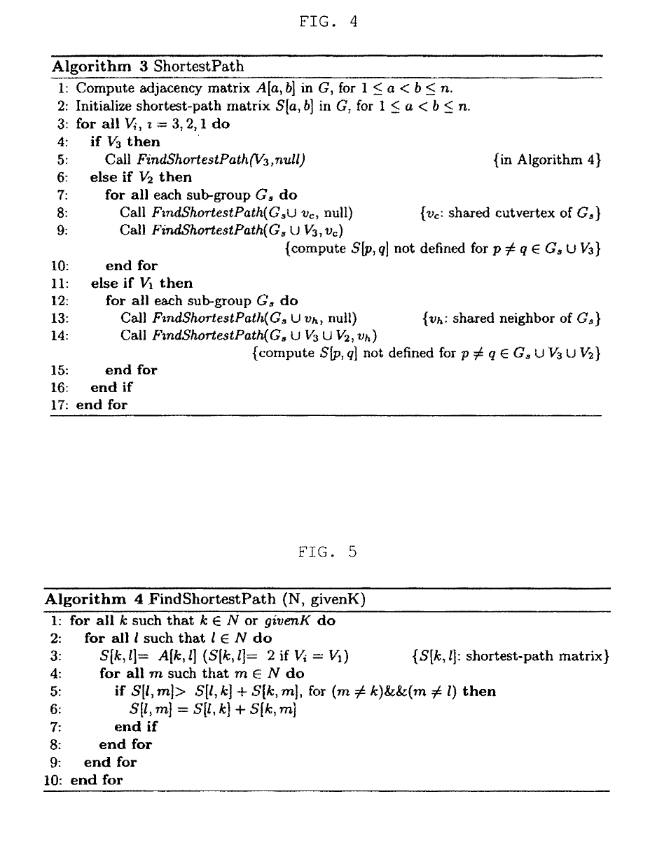

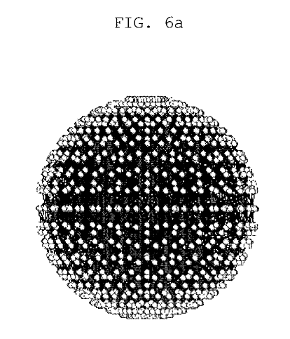

In accordance with the present invention, with reference to FIGS. 4 and 5, there is provided a way to find shortest paths in each group. As shown in FIGS. 4 and 5 describing an algorithm computing shortest paths, a shortest path between every pair of nodes is computed for each group Vᵢ (i=1, 2, 3). For V₂ and V₁, shortest paths are determined in each of their sub-groups. After computing shortest paths between nodes in each sub-group, shortest paths between nodes of V₂ and nodes of V₃ are computed using a shared cutvertex of each sub-group of V₂ (line 9 in FIG. 4). Likewise, shortest paths between nodes of V₁ and nodes of V₂ and V₃ are computed using a shared neighboring node of each sub-group of V₁ (line 14 in FIG. 4). For sub-groups of V₁, an initial shortest path between every pair of nodes is set to 2, since the distance between a node and its shared neighbor is 1 (line 3 in FIG. 5).

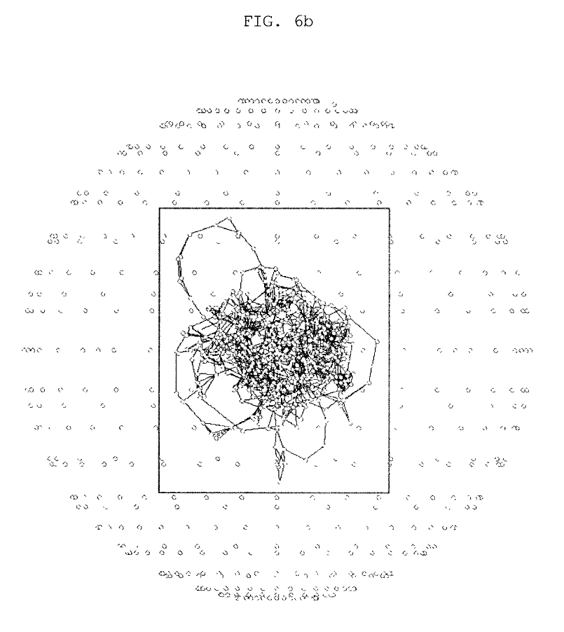

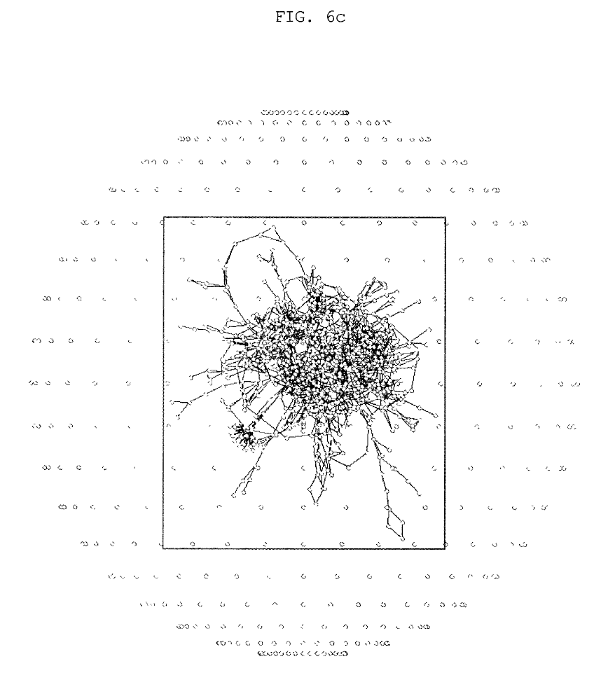

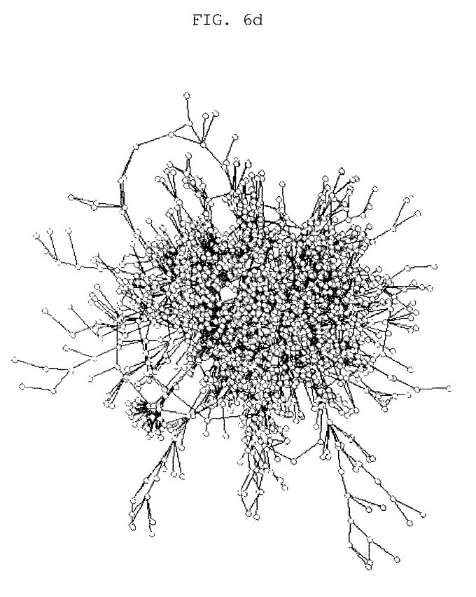

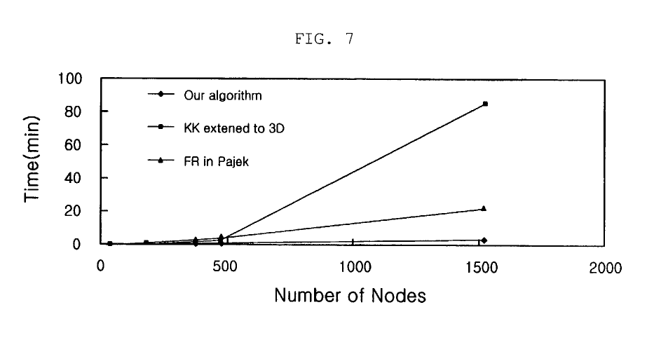

FIGS. 6a to 6d illustrate a drawing process of MIPS physical interaction data (MIPS-P). FIG. 6a shows an initial layout by the algorithm according to the present invention for MIPS physical interaction data with 1526 nodes and 2372 edges. The graphs after drawing nodes of V₃ in a rectangle, and drawing nodes of V₂ and V₃ in the rectangle, are shown in FIGS. 6b and 6c, respectively. Also, FIG. 6d shows a final drawing. While groups are determined in the order of V₁, V₂ and V₃, their layout is performed in reverse order. V₃ is first positioned in the center of a sphere, V₂ in the outer region of V₃, and V₁ then in the outer region of V₂ and V₃. Groups in which node positions are fixed are shown in the rectangle. Nodes in the remaining groups are relocated with modified polar coordinates to place the outer region of the groups that have been fixed. In FIGS. 6b and 6c, edges between nodes in the outer region not drawn for clear drawing. Nodes in each group are positioned using a spring-force layout, for which shortest paths are computed according to the algorithms in FIGS. 4 and 5.

The computational cost of the algorithm for visualizing protein interaction data according to the present invention is analyzed as follows. Assuming that three groups are balanced, total time for the algorithm according to the present invention is

$${( \frac{n}{3} )}^{3} + {( \frac{n}{3} )}^{3} + {( \frac{n}{3} )}^{3} = \frac{{n}^{3}}{9}$$

because a spring-embedder algorithm is applied to each group. The asymptotic time complexity of the algorithm according to the present invention is the same as the time complexity O (n³) of Kamada & Kawai's algorithm. However, the algorithm according to the present invention is practically much faster than Kamada & Kawai's algorithm. Since nodes of V1 and V2 are further divided into sub-groups, actual running time is further reduced for the graph with balanced groups. For graphs with unbalanced groups (for example, graphs in which the portion of V3 is high owing to few cutvertices and terminal nodes), the effect of dividing nodes into three groups can be marginal, and this phenomenon is rare in protein interaction data. This fact is supported by the experimental result, as will be described, below.

The algorithm according to the present invention was implemented in Microsoft's C#. The program runs on any PC with Windows 2000/XP/Me/98/NT 4.0 as its operating system. The test was performed using the program for five cases, Brain (http://www.infosun.fmi.uni-passau.de/GD2001/qraphC/brain.gml), Gd29 (http://www.infosun.fmi.uni-passau.de/GD2001/graphA/GD29.gml), Y2H, and genetic and physical interaction data from the MIPS database (http://mips.gsf.de/proj/yeast/tables/interaction). In protein interaction data from Y2H and MIPS, the largest connected components were used.

Table 1 shows running times of the algorithm according to the present invention at each stage of partitioning nodes into three groups (P), finding shortest paths in each group (SP), and layout and drawing (LD). The test cases of Brain and Gd29 are different from the others, which are protein interaction data, in the size of data sets as well as in the relative size of their V₃. In case of Brain, 28 (84.8%) of total 33 nodes belong to V3, and in case of Gd29, 128 (71.9%) of total 178 nodes belong to V3. However, the ratio of V3 to the total number of nodes was less than 50% in cases of Y2H, MIPS-G and MIPS-P (24.9%, 43.5% and 37.4%, respectively).

TABLE 1

| | | Nodes | | | Running times | | | |
|---|---|---|---|---|---|---|---|---|
| Data | Edges | V1 | V2 | V3 | P | SP | LD | Total = (P + SP + LD) |
| Brain | 135 | 4 | 1 | 28 | 0.08 s | 0.02 s | 0.15 s | 0.25 s |
| Gd29 | 344 | 40 | 10 | 128 | 0.84 s | 0.90 s | 2.06 s | 3.80 s |
| Y2H | 542 | 255 | 100 | 118 | 1.41 s | 0.87 s | 3.49 s | 5.77 s |
| MIPS-G | 805 | 198 | 102 | 231 | 3.24 s | 5.16 s | 8.52 s | 16.92 s |
| MIPS-P | 2372 | 665 | 289 | 572 | 56.39 s | 1 m 18.82 s | 56.20 s | 3 m 11.41 s |

As described hereinbefore, the method for partitioned layout of protein interaction networks according to the present invention yields a clear and aesthetically pleasing drawing for large-scale protein interaction networks as shown in FIG. 6, and is much faster than other forced-directed layouts.

For experimental comparison with the conventional algorithms, Pajek with Fruchterman & Reingold's algorithm and the extended Kamade & Kawai's algorithm were run. Because of producing only a two-dimensional drawing, Kamade & Kawai's algorithm was extended into a three-dimensional drawing. Table 2, below, shows running times of the algorithm according to the present invention, Kamade & Kawai's algorithm extended to 3D, and Fruchterman & Reingold's algorithm (Pajek(F-R)) on the five test cases on a Pentium II 299 Mhz processor. As shown in Table 2, with the partitioning method according to the present invention the computation time was found to be significantly reduced by up to 51 times. Also, the resulting data is shown in a graph in FIG. 7 comparing running times of three algorithms, demonstrating that the algorithm according to the present invention is more effective for bigger graphs and for graphs not having an excessively high proportion of V₃.

TABLE 2

| | The algorithm of the present | K—K extended to | |
|---|---|---|---|
| Data | invention | 3D | Pajek (F-R) |
| Brain | 0.25 s | 0.19 s | 7.57 s |
| Gd29 | 3.80 s | 4.77 s | 25.28 s |
| Y2H | 5.77 s | 1 m 23.46 s | 2 m 23.32 s |
| MIPS-G | 16.92 s | 1 m 50.62 s | 3 m 18.35 s |
| MIPS-P | 3 m 11.41 s | 1 h 24 m 42.12 s | 21 m 41.91 s |

1. A method for partitioned layout of protein interaction networks, which yields a graph using proteins as nodes and interactions between proteins as edges to visualize protein interaction data, comprising the steps of:
grouping nodes into group 1, which is a set of terminal nodes with degree 1, group 2, which is a set of nodes in subgraphs containing a small number of nodes among subgraphs separated by cutvertices, except nodes of group 1, and group 3, consisting of nodes which are members of neither group 1 nor 2;
computing shortest paths between nodes of each group, shortest paths between nodes of said group 1 and nodes of said group 2, shortest paths between nodes of said group 1 and nodes of said group 3, and shortest paths between nodes of said group 2 and nodes of said group 3; and
performing layout by positioning nodes of said group 3 in the center of a sphere, nodes of said group 2 in the outer region of said group 3, and nodes of said group 1 in the outer region of said groups 2 and 3, by spring-force layout algorithm using said shortest paths.
